(19) United States
(12) Patent Application Publication
(10) Pub. No.: US 2019/0062293 A1
(43) Pub. Date: Feb. 28, 2019

(54) METAL-ORGANIC FRAMEWORKS FOR THE CONVERSION OF LIGNOCELLULOSIC DERIVATIVES TO RENEWABLE PLATFORM CHEMICALS

(71) Applicants: Noppadon Sathitsuksanoh, Louisville, KY (US); Mark D. Allendorf, Pleasanton, CA (US); Anthe George, San Francisco, CA (US); Robert Jansen, Collinsville, IL (US); Kirsty Leong, Pleasanton, CA (US); Blake A. Simmons, San Francisco, CA (US); Seema Singh, Fremont, CA (US); Philip Travisano, III, Danville, VA (US)

(72) Inventors: Noppadon Sathitsuksanoh, Louisville, KY (US); Mark D. Allendorf, Pleasanton, CA (US); Anthe George, San Francisco, CA (US); Robert Jansen, Collinsville, IL (US); Kirsty Leong, Pleasanton, CA (US); Blake A. Simmons, San Francisco, CA (US); Seema Singh, Fremont, CA (US); Philip Travisano, III, Danville, VA (US)

(21) Appl. No.: 15/539,122

(22) PCT Filed: Dec. 17, 2015
(86) PCT No.: PCT/US2015/066474

§ 371 Date: Jun. 22, 2017

Related U.S. Application Data
(60) Provisional application No. 62/096,455, filed on Dec. 23, 2014.

(51) Int. Cl. C07D 307/50; B01J 31/16

(52) U.S. Cl. CPC C07D 307/50; B01J 31/1691

(57) ABSTRACT

Methods of processing lignocellulose using metal-organic frameworks (MOFs) to form renewable platform chemicals that may be used as initial feedstock are provided. Metal-organic frameworks react with Provide lignocellulosic derivative lignocellulosic derivatives such as glucose and fructose to form 5-hydroxymethyl furfural (HMF) with high yield and high selectivity for FIMF production.

### REFERENCE TO RELATED APPLICATIONS

This application claims the benefit of U.S. Provisional Application Ser. No. 62/096,455, filed on Dec. 23, 2014, the entire contents of which are hereby incorporated by reference.

### STATEMENT OF GOVERNMENTAL SUPPORT

This invention was made with government support under Contract No. DE-ACO2-05CH11231 awarded by the U.S. Department of Energy. The government has certain rights in the invention.

### BACKGROUND

Biomass resources are used in various industrial applications to provide renewable energy sources. Cellulose from lignocellulose is the most abundant bioresource on the planet. Cellulose includes glucose building blocks, which may be converted to other derivatives for use as a biofuel and chemicals in various industries.

### SUMMARY

Provided herein are methods of processing lignocellulose. One aspect involves a method of processing lignocellulose including converting lignocellulosic derivatives to 5-hydroxymethyl furfural by reacting the lignocellulosic derivatives with a metal organic framework.

In some embodiments, the metal organic framework comprises aluminum. In some embodiments, the metal organic framework comprises MIL-101.

In various embodiments, the lignocellulosic derivatives comprise glucose. In some embodiments, the lignocellulosic derivatives comprise fructose.

The method may include prior to converting lignocellulosic derivatives to 5-hydroxymethyl furfural, forming the lignocellulosic derivatives from feedstock. In some embodiments, forming lignocellulosic derivatives includes acidolysis of cellulose to glucose. In some embodiments, forming lignocellulosic derivatives includes converting glucose to fructose using glucose isomerase and a borate salt.

The method may be performed in an acidic ionic liquid solvent. The acidic ionic liquid solvent may be any of [C2mim]Cl, [C3mim]Cl, and [C4mim]Cl.

In some embodiments, forming the lignocellulosic derivatives and converting the lignocellulosic derivatives are performed in the same acidic ionic liquid solvent.

The percent yield of 5-hydroxymethyl furfural may be at least about 5% in weight. In some embodiments, the selectivity for 5-hydroxymethylfurfural is at least about 70%.

These and other aspects are described further below with reference to the drawings.

### BRIEF DESCRIPTION OF THE DRAWINGS

FIG. 1 depicts reaction pathways for forming 5-hydroxymethyl furfural from cellulose.

FIG. 2 is a process flow diagram depicting operations for performing a method in accordance with disclosed embodiments.

FIG. 3 depicts reactions for conversions between glucose and fructose.

FIGS. 4-6 are graphs of experimental results from performing methods in accordance with disclosed embodiments.

FIG. 7 depicts examples of metal organic frameworks that may be used in accordance with disclosed embodiments.

### DETAILED DESCRIPTION

In the following description, numerous specific details are set forth to provide a thorough understanding of the presented embodiments. The disclosed embodiments may be practiced without some or all of these specific details. In other instances, well-known process operations have not been described in detail to not unnecessarily obscure the disclosed embodiments. While the disclosed embodiments will be described in conjunction with the specific embodiments, it will be understood that it is not intended to limit the disclosed embodiments.

The production of biofuels and chemicals provides resources to various industries. Biofuels may be formed by biomass conversion, or in particular, lignocellulosic biomass conversion. Example types of lignocellulosic biomass include aromatic polymers, such as lignin, and carbohydrate polymers, such as cellulose and hemicellulose.

Cellulose from lignocellulose is the most abundant bioresource on the planet and processes to convert cellulose into compounds may be suitable for use in many industries. In particular, cellulose may be used as a feedstock to form other compounds. Cellulose consists mainly of glucose building blocks. Various industries use conversion of glucose to other compounds as a precursor to producing chemicals and materials in industrial processes.

The production of fuels and chemicals from lignocellulose-derived 5-hydroxymethyl furfural (HMF) is of particular interest, since HMF can be further converted to C₉-C₁₅ alkanes, 2,5-dimethylfuran, ethyl levulinate, 5-(alkoxymethyl)furfurals, and 2,5-bis(alkoxymethyl)furans. Conversion of glucose to HMF may occur via a dehydration reaction, or via formation of an intermediate such as fructose.

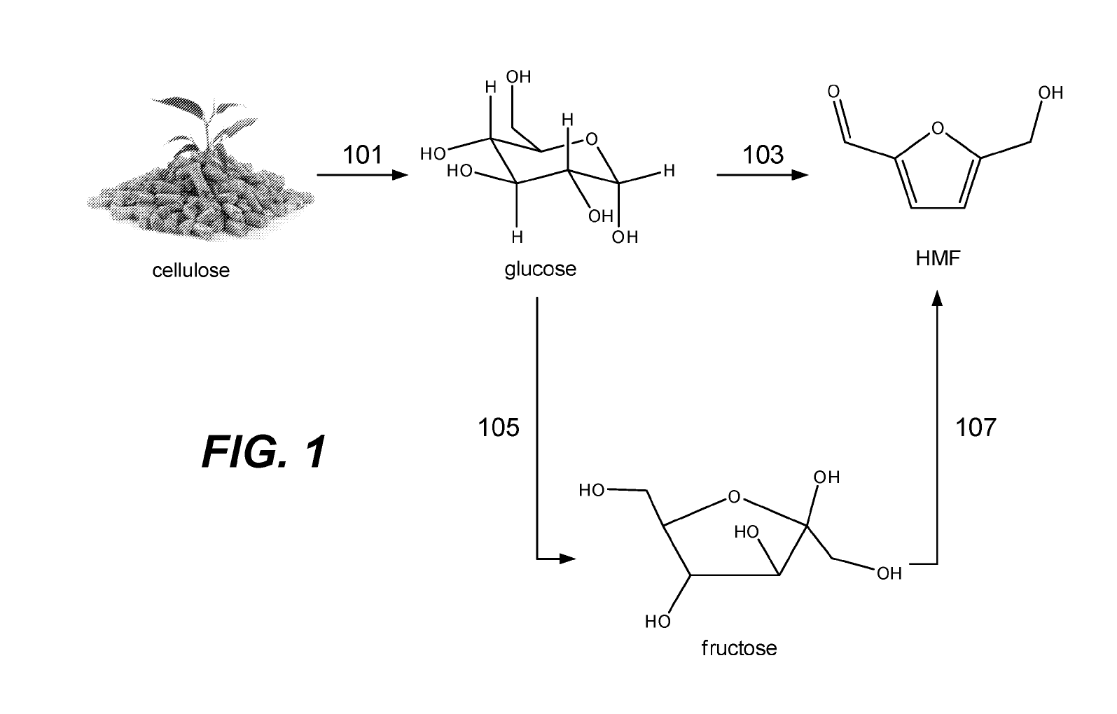

FIG. 1 depicts example pathways for forming HMF. As shown, cellulose may break down into glucose via reaction 101, a hydrolysis reaction. After glucose is formed, glucose may undergo reaction 103 to directly form HMF—this reaction includes a dehydration mechanism. Alternatively, glucose may undergo reaction 105 to form fructose as an intermediate. This may be performed by isomerization of glucose. Subsequently, fructose may undergo reaction 107 to form HMF, the reaction of which may include dehydration. In various processes, the conversion from glucose to HMF via reactions 105 and 107 may occur quickly such that fructose may go undetected.

In some processes, humin by-products may be formed. Humins may be heterogeneous undesired waste. For example, humins may be formed in the conversion from cellulose to glucose, or glucose to fructose, or glucose to HMF, or fructose to HMF. The amount of material A that is converted in a reaction may have reacted to form waste by-products or other compounds in addition to a desired product. As used herein, a percent conversion or percent converted of a material A is defined as the amount of A that reacted or converted divided by initial amount of A used in the reaction. Thus, the percent conversion includes production of desired products as well as production of humins.

In processes described herein, the selectivity for a reaction mechanism to form a specific product may be determined. In a reaction where A is reacted to form B, and B is the specific, desired product, the reaction may also form some other by-products C. In some reactions, some of A may be unreacted, such that a post-reaction mixture includes B, C, and some A. The selectivity of a chemical or reaction mechanism is defined as the amount of B, a specific product, divided by the amount of A reacted to form a new product, desired or otherwise; referred to above as converted. That is, selectivity of a specific product may be defined as how much specific product is formed from the total amount of the initial reactant that converted. A higher selectivity indicates that there is less undesirable product formation.

As an example, a reaction may include converting A to B using a catalyst, with some excess by-product C:

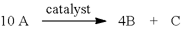

In such an example, 10 moles of A may be mixed with a catalyst to form 4 moles of B and 1 mole of C. If the resulting mixture of A, B, and C includes 2 moles of A, then only 8 moles of A was converted or reacted in the reaction. Thus, the percent conversion of A is:

$$Percent Conversion of A = \frac{8 moles converted}{10 moles initial} \times 100 \% = 80 \%$$

If the resulting mixture of A, B, and C includes 4 moles of B after having converted 8 moles of A, then the selectivity of B is:

$$Selectivity of B = \frac{4 moles of B product}{8 moles of A converted} \times 100 \% = 50 \%$$

Note that as a result, processes described herein may focus on maximizing selectivity rather than maximizing percent conversion because even if percent conversion of A is high, if selectivity to form B is low, then the process efficiency for obtaining B is low, as a substantial amount of A may be converted to a waste by-product C (e.g., a humin) from which it is not possible to generate the desired product B further. If percent conversion of A is low, but selectivity to form B is high, then the process efficiency for obtaining B is high, since the amount of A that is not converted may be recycled and used in the process again to form B. A higher selectivity of B suggests less by-products C are formed, so high selectivity is useful in achieving a more efficient and economical process.

Provided herein are methods of processing lignocellulose using metal-organic frameworks as catalysts to achieve high selectivity for the formation of HMF. In some embodiments, HMF selectivity is at least about 73 mol %. Such methods can achieve a high yield of HMF in one or more cycles of the process, which is scalable to industrial uses. In some embodiments, the chemical pathway forms HMF from glucose without separately forming a fructose intermediate.

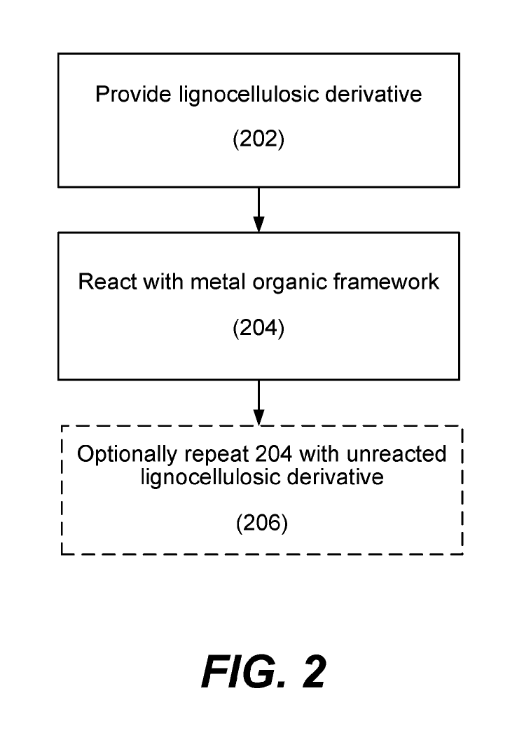

FIG. 2 is a process flow diagram depicting operations for a method in accordance with disclosed embodiments. In operation 202, a lignocellulosic derivative is provided, for example, to a stirred tank reactor. The lignocellulosic derivative may be formed by converting cellulose via an acidolysis or acid hydrolysis mechanism. For example, cellulose may be pretreated with a solvent and reacted with an acid such as HCl over time (such as about 1 hour) to slowly convert cellulose to glucose without polymerizing the lignocellulosic derivative. In one example, about 15 grams of feedstock is pretreated in a solvent of 1-n-butyl-3-methylimidazolium chloride ([C₄mim]Cl) at 140° C. for an hour, and the cellulose undergoes acid hydrolysis in 85 g of [C₄mim]Cl slowly over 1 hour while maintaining a low pH of about 1. The lignocellulosic derivative may be a monosaccharide, or in some embodiments, an oligosaccharide or polysaccharide. In various embodiments, the lignocellulosic derivative is glucose. In some embodiments, conversion of cellulose to glucose may achieve a glucose yield between about 93 wt % and 96 wt %. In various embodiments, the solvent used to break down biomass to a lignocellulosic derivative completely dissolves the biomass. In some examples, the solvent is [C₄mim]Cl.

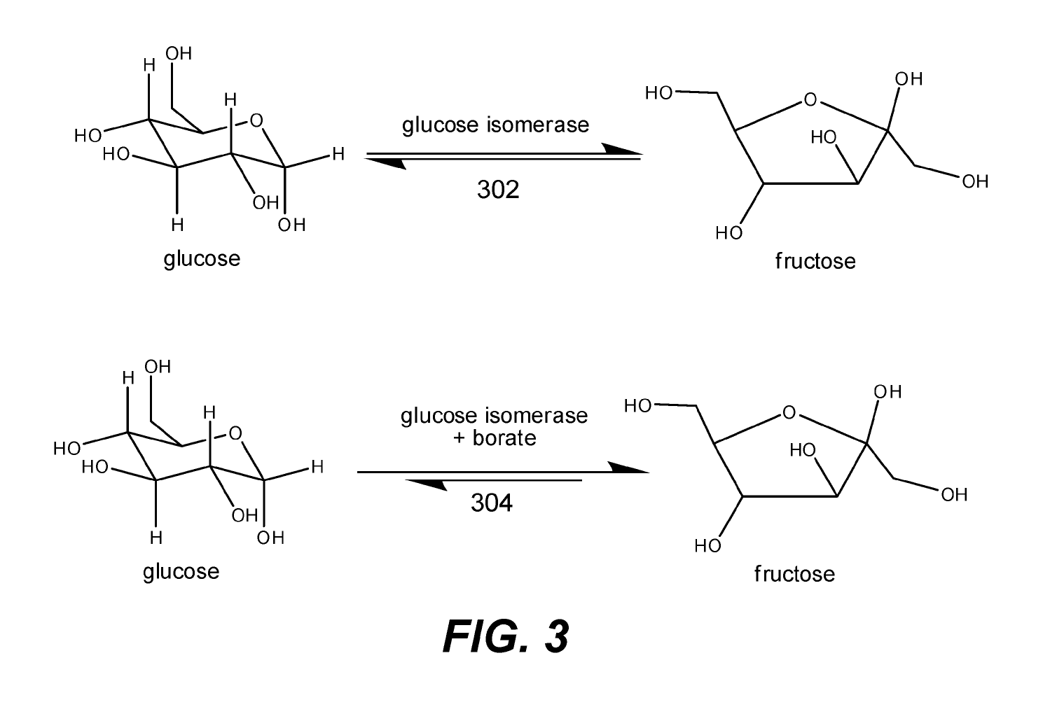

In various embodiments, the lignocellulosic derivative is fructose. Fructose may be formed by converting glucose via an enzymatic pathway. FIG. 3 shows a reaction 302 whereby glucose isomerase catalyzes conversions between glucose and fructose. Since the reaction involving glucose isomerase is a reversible reaction and glucose and fructose are isomers of each other, equal amounts of glucose and fructose are present at equilibrium. That is, a reaction mixture that starts with glucose and catalyzed by glucose isomerase may form a mixture with 50% glucose and 50% fructose. In some embodiments, reaction 304 may be used instead of reaction 302 to yield more fructose. That is, a borate salt such as boric acid (H₃BO₃) or sodium borate (Na₂B₄O₇.10H₂O) may be added to the reaction mixture. Without being bound by a particular theory, borate salts may form a complex with fructose, thereby hampering glucose isomerase's function to isomerize fructose back to glucose. In some embodiments, adding sodium borate may form about 70% fructose and about 30% glucose.

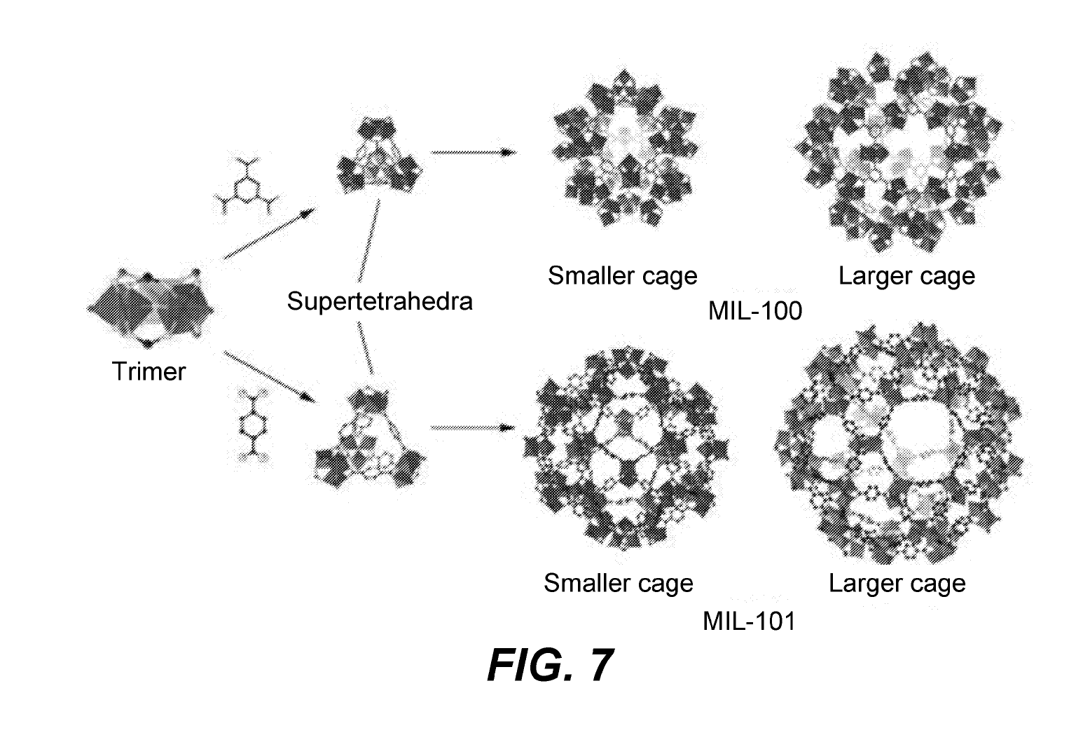

Returning to FIG. 2, in operation 204, metal organic frameworks (MOFs) are reacted with the lignocellulosic derivative to form HMF. MOFs are a class of heterogeneous solid catalysts that may be suitable for use in aqueous systems. A MOF is a coordination network with organic ligands linking metal ions or clusters. A MOF may be a trimer, a supertetrahedra, a cage structure, or another coordination structure. FIG. 7 shows example structures of MOFs, whereby the dark blocks are metal sites and the thinner bonds are organic compounds linking the metal sites together. A discussion of the structures and preparation of MOFs is described in “Conversion of Fructose into 5-hydroxymethylfurfural Catalyzed by Recyclable Sulfonic Acid-functionalized Metal-organic Frameworks” by Chen, Jinzhu et al. (Green Chem., 2014, 16. 2490-2499), which is herein incorporated by reference in its entirety. Without being bound by a particular theory, it is believed that the mole ratio of metal sites to the lignocellulosic derivative drives the reaction to form HMF.

In various embodiments, the MOFs used in operation 204 are capable of catalyzing both glucose and fructose. For example, MOFs including MIL-101 (Materials of Institute Lavoisier) may be used in disclosed embodiments. In various embodiments, MOFs used in operation 204 are bifunctional catalysts. In some embodiments, MOFs converting glucose directly to HMF may act as both an isomerization catalyst to form fructose and a dehydration catalyst to convert fructose to HMF.

MOFs may include open metal sites located on the outer surface of the MOF structure, or open metal sites located inside a MOF structure. An open metal site in MOFs is defined as an uncoordinated bond on a metal atom of the MOF. For example in MIL-101-Al—NH₂, the Al atom has an available binding site that can bond to solvent molecules or other species, such as glucose and fructose, to catalyze the HMF production reaction. A large cage formation may have a large surface area such that there are many open metal sites on the outer surface of the structure. For example, the maximum Langmuir surface area of a MOF used in disclosed embodiments may be greater than about 1600 m²/g. In some embodiments, the MOFs used in operation 204 may have a maximum surface area of about 5900 m²/g. Without being bound by a particular theory, the metal sites on a MOF may be the reactive sites on the MOFs that drive the conversion reaction from the lignocellulosic derivative to HMF. In larger compounds, such as the larger cage MIL-101 in FIG. 7, MOFs may include pores. In some embodiments, pores may be large enough such that even if open metal sites are on the inner surface of the MOF structure, a molecule such as glucose or fructose may freely enter a MOF through the pores to react with inner open metal sites.

MOFs are solid compounds and therefore may be separated more easily from an aqueous mixture. Thus, a lignocellulosic derivative from operation 202 may be in aqueous form, and upon reacting with a MOF to form HMF, any excess MOF may be extracted and reused in subsequent processes or cycles.

MOFs suitable for use in disclosed embodiments may include aluminum, chromium, and zirconium. In some embodiments, MOFs may be functionalized with an amine group. In some embodiments, the MOFs may be functionalized with a sulfonic acid. Examples of sulfonic acid-functionalized MOFs used in carbohydrate valorization are described in “Conversion of Fructose into 5-hydroxymethylfurfural Catalyzed by Recyclable Sulfonic Acid-functionalized Metal-organic Frameworks” by Chen, Jinzhu et al. (Green Chem., 2014, 16. 2490-2499), which is herein incorporated by reference in its entirety.

During operation 204, an ionic liquid (IL) solvent may be used, such as 1-butyl-3-methylimidazolium chloride ([C₄mim][Cl]). Other suitable solvents include 1-ethyl-3-methylimidazolium chloride ([C₂mim]Cl), 1-propyl-3-methylimidazolium chloride ([C₃mim]Cl), and other acidic ionic liquids. Other suitable IL that can be used in the disclosed embodiments include any IL that does not impede the forming of HMF. In some embodiments, the IL is also suitable for pretreatment of biomass and for the hydrolysis of cellulose by thermostable cellulase. Suitable IL are taught in ChemFiles (2006) 6(9) (which are commercially available from Sigma-Aldrich; Milwaukee, Wis.). Such suitable IL include, 1-alkyl-3-alkylimidazolium alkanate, 1-alkyl-3-alkylimidazolium alkylsulfate, 1-alkyl-3-alkylimidazolium methylsulfonate, 1-alkyl-3-alkylimidazolium hydrogensulfate, 1-alkyl-3-alkylimidazolium thiocyanate, and 1-alkyl-3-alkylimidazolium halide, where an “alkyl” is an alkyl group including from 1 to 10 carbon atoms, and an “alkanate” is an alkanate including from 1 to 10 carbon atoms. In some embodiments, the “alkyl” is an alkyl group including from 1 to 4 carbon atoms. In some embodiments, the “alkyl” is a methyl group, ethyl group or butyl group. In some embodiments, the “alkanate” is an alkanate including from 1 to 4 carbon atoms. In some embodiments, the “alkanate” is an acetate. In some embodiments, the halide is chloride.

Additional suitable IL include, but are limited to, 1-ethyl-3-methylimidazolium acetate (EMIM Acetate), 1-ethyl-3-methylimidazolium chloride (EMIM Cl), 1-ethyl-3-methylimidazolium hydrogensulfate (EMIM HOSO₃), 1-ethyl-3-methylimidazolium methyl sulfate (EMIM MeOSO₃), 1-ethyl-3-methylimidazolium ethyl sulfate (EMIM EtOSO₃), 1-ethyl-3-methylimidazolium methanesulfonate (EMIM MeSO₃), 1-ethyl-3-methylimidazolium tetrachloroaluminate (EMIM AlCl₄), 1-ethyl-3-methylimidazolium thiocyanate (EMIM SCN), 1-butyl-3-methylimidazolium acetate (BMIM Acetate), 1-butyl-3-methylimidazolium chloride (BMIM Cl), 1-butyl-3-methylimidazolium hydrogensulfate (BMIM HOSO₃), 1-butyl-3-methylimidazolium methanesulfonate (BMIM MeSO₃), 1-butyl-3-methylimidazolium methylsulfate (BMIM MeOSO₃), 1-butyl-3-methylimidazolium tetrachloroaluminate (BMIM AlCl₄), 1-butyl-3-methylimidazolium thiocyanate (BMIM SCN), 1-ethyl-2,3-dimethylimidazolium ethyl sulfate (EDIM EtOSO₃), Tris(2-hydroxyethyl)methylammonium methylsulfate (MTEOA MeOSO₃), 1-methylimidazolium chloride (MIM Cl), 1-methylimidazolium hydrogensulfate (MIM HOSO₃), 1,2,4-trimethylpyrazolium methylsulfate, tributylmethylammonium methylsulfate, choline acetate, choline salicylate, and the like. The ionic liquid can include one or a mixture of the compounds. Further ILs are described in U.S. Pat. No. 6,177,575 (which is herein incorporated by reference), which describes ILs having the following structure:

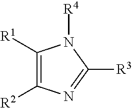

whereby R¹, R² and R³ are each independently hydrogen, hydrocarbyl or substituted hydrocarbyl; and R⁴ is hydrogen, alkyl, or substituted alkyl.

In some embodiments, the solvent used in operation 204 is the same as the solvent used to provide the lignocellulosic derivative in operation 202. For example, in some embodiments, biomass may be converted to the lignocellulosic derivative in the solvent, and MOFs may then be added to the mixture to convert the lignocellulosic derivative to HMF. In various embodiments, an acidic ionic liquid is used such that acidolysis of biomass such as cellulose may convert to glucose. In some embodiments where the lignocellulosic derivative is provided in operation 202 without first performing acidolysis of cellulose, the ionic liquid solvent may be nonacidic.

Operation 204 may be performed at a temperature between about 100° C. and about 120° C., depending on the lignocellulosic derivative used. Mixtures may be reacted for a time between about 20 minutes and about 120 minutes, depending on the lignocellulosic derivative used.

Resulting selectivity to HMF may be at least about 80 mol %, such as about 87.6 mol %. In some embodiments, selectivity to HMF is at least about 60% wt %, such as about 61.3 wt %. In some embodiments, the percent of lignocellulosic derivative converted may range widely, such as between about 5% and about 75%.

In some embodiments where an aluminum-containing MOF is reacted with a lignocellulosic derivative, conversion may be between about 5% and about 15%, with HMF selectivity of at least about 70%, or at least about 75%. This selectivity suggests that little waste or humin by-products are formed in the reaction. However, since percent conversion is low, the process may be cycled to maximize HMF formation. Thus, in operation 206, operation 204 is optionally repeated by reacting unreacted or unconverted lignocellulosic derivative with MOFs to form HMF. In some embodiments, this operation involves extracting HMF from the reaction mixture, and reacting MOF with the lignocellulosic derivative to drive formation of HMF.

In some embodiments, the HMF yield in weight percent from converting glucose using an MOF may be at least about 4 wt %, or at least about 6 wt %, or at least about 9 wt %. In some embodiments, the HMF yield in weight percent from converting fructose using an MOF may be at least about 6 wt %, or at least about 10 wt %, or at least about 30 wt %. In some embodiments, a HMF yield of about 55 wt % HMF was achieved. For example, for a method performed at about 120° C. for about 1 hour for converting glucose to HMF, the HMF selectivity was at least about 78.63 mol % HMF which suggests that if 100% glucose is dehydrated by MIL-101-Al—NH₂, 78.63 mol % HMF may be obtained, which translates to about 55 wt % HMF.

The HMF yield in mole percent for converting glucose using an MOF may be at least about 5 mol %, or at least about 9 mol %, or at least about 14 mol %. For example, HMF yield from converting glucose may be about 14.24 mol %. The HMF yield in mole percent for converting fructose using an MOF may be at least about 7 mol %, or at least about 15 mol %, or at least about 40 mol %. For example, HMF yield from converting fructose may be about 47.09 mol %.

Experimental

Experiment 1: Glucose Isomerization

An experiment was conducted to evaluate the effect of using a borate salt in a conversion reaction between glucose and fructose. Five trials were conducted, each having varying amounts of sodium borate. In each trial, 600 mg of glucose was mixed with 18 mg of Sweetzyme®, a ready-immobilized glucose isomerase available from Novozymes of Denmark and 10 mg of magnesium sulfate (MgSO₄) in 5 mL of water H₂O at a temperature of 70° C. in a 500 mL stirred tank reactor such as 500 mL HP/HT reactors from the 4570 Series, available from Parr Instrument Company of Moline, Ill. The first trial did not use sodium borate, and the subsequent trials used borate such that the glucose to borate ratios by molar ratio were 1:2, 1:1, 1:0.5, and 1:0.25. The results are summarized in Table 1 below.

TABLE 1
Effect of borate on glucose isomerization

| Glucose | | | | | | | | | |
|---|---|---|---|---|---|---|---|---|---|
| to Borate | | | Time (hr) | | | | | | |
| Ratio | | | 2 | 4 | 6 | 8 | 10 | 24 | 52 |
| 1:2 | Conv. | (%) | 1.42 | 7.86 | 17.85 | 17.53 | 16.57 | 38.54 | 60.62 |
| | Fructose | (%) | 6.58 | 9.69 | 11.25 | 13.08 | 14.5 | 14.39 | 11.06 |
| | Selectivity | (%) | 463.0 | 123.4 | 63.1 | 74.6 | 87.5 | 37.3 | 18.3 |
| 1:1 | Conv. | (%) | 22.88 | 23.18 | 31.39 | 39.89 | 41.85 | 66.69 | 71.00 |
| | Fructose | (%) | 13.71 | 25.19 | 33.08 | 37.36 | 42.71 | 40.81 | 27.89 |
| | Selectivity | (%) | 59.9 | 108.7 | 105.4 | 93.7 | 102.1 | 61.2 | 39.3 |
| 1:0.5 | Conv. | (%) | 27.34 | 48.28 | 62.25 | 69.49 | 75.41 | 79.59 | 88.47 |
| | Fructose | (%) | 26.48 | 45.26 | 57.34 | 65.29 | 70.66 | 79.59 | 61.76 |
| | Selectivity | (%) | 96.9 | 93.7 | 92.1 | 94.0 | 93.7 | 100 | 69.8 |
| 1:0.25 | Conv. | (%) | 39.32 | 63.64 | 74.37 | 78.23 | 78.3 | 77.11 | 81.39 |
| | Fructose | (%) | 33.84 | 52.32 | 60.00 | 63.05 | 70.47 | 77.11 | 64.03 |
| | Selectivity | (%) | 86.1 | 82.2 | 80.7 | 80.6 | 90.0 | 100.0 | 78.7 |
| no borate | Conv. | (%) | 37.32 | 44.97 | 50.54 | 47.26 | 47.11 | 43.34 | 51.67 |
| | Fructose | (%) | 30.82 | 39.24 | 37.47 | 40.29 | 40.53 | 43.34 | 37.2 |
| | Selectivity | (%) | 82.6 | 87.2 | 74.1 | 85.2 | 86.0 | 100.0 | 72.0 |

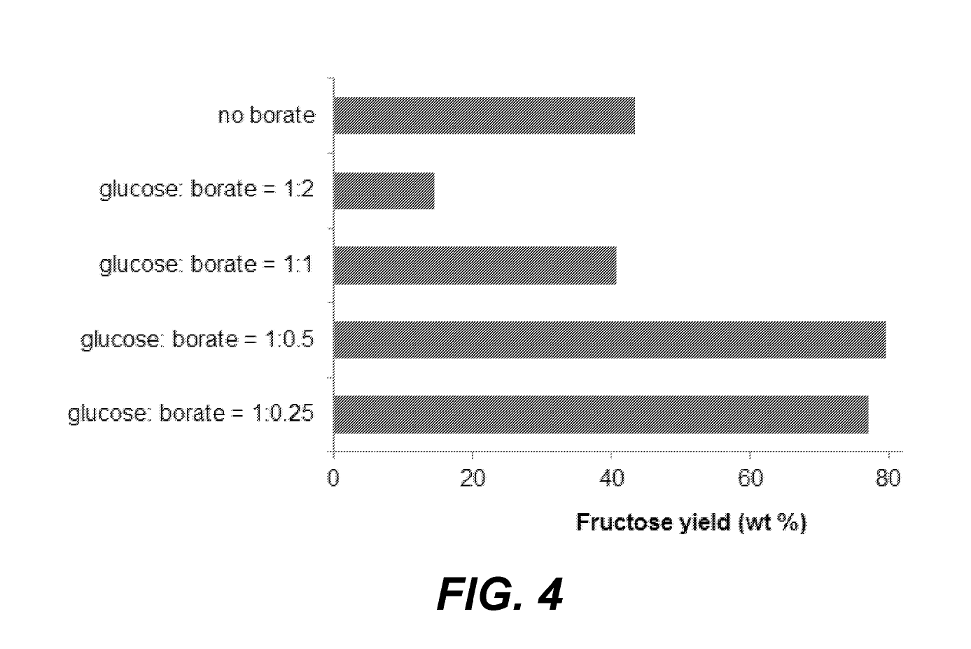

Table 1 shows the percent conversion of glucose to fructose, fructose yield, and selectivity for fructose in each trial. FIG. 4 shows a bar graph depicting fructose yield for each trial. As shown, using a glucose to borate ratio of 1:0.5 and 1:0.25 yielded the most amount of fructose—that is, the reaction mixture had more fructose than glucose, suggesting the borate salt may have interacted with the mixture such that glucose isomerase is hindered from converting fructose back to glucose. Selectivity for fructose was also particularly high at these ratios (over 80%).

The effect of pH on glucose isomerization with a glucose to borate ratio of 1:0.5 was evaluated for pH at 4, 5, 6, 7, and 8. The results are summarized in Tables 2A, 2B, and 2C below. In this experiment, pH from 4-8 had little to no effect on glucose conversion and fructose selectivity.

TABLE 2A
Glucose Isomerization at pH = 4, 5

| | pH 4 | | | pH 5 | | |
|---|---|---|---|---|---|---|
| Time | Conv. | Yield | Sel. | Conv. | Yield | Sel. |
| (hr) | (%) | (%) | (%) | (%) | (%) | (%) |
| 2 | 31.3 | 28.1 | 89.7 | 28.6 | 30.2 | 105.4 |
| 4 | 50.2 | 49.2 | 98.1 | 51.9 | 48.1 | 92.7 |
| 6 | 66.1 | 59.1 | 89.5 | 66.5 | 59.7 | 89.7 |
| 8 | 73.4 | 66.2 | 90.2 | 72.9 | 69.4 | 95.3 |
| 10 | 78.5 | 70.8 | 90.2 | 78.5 | 73.4 | 93.6 |
| 24 | 80.3 | 80.3 | 100.0 | 80.7 | 80.7 | 100.0 |
| 52 | 88.5 | 57.3 | 64.8 | 88.1 | 61.1 | 69.4 |

TABLE 2B
Glucose Isomerization at pH = 6, 7

| | pH 6 | | | pH 7 | | |
|---|---|---|---|---|---|---|
| Time | Conv. | Yield | Sel. | Conv. | Yield | Sel. |
| (hr) | (%) | (%) | (%) | (%) | (%) | (%) |
| 2 | 26.6 | 31.8 | 119.3 | 25.0 | 33.8 | 135.1 |
| 4 | 48.2 | 55.4 | 114.9 | 53.2 | 52.3 | 98.3 |
| 6 | 65.5 | 66.3 | 101.3 | 67.5 | 65.1 | 96.4 |
| 8 | 73.0 | 74.0 | 101.4 | 75.3 | 69.6 | 92.4 |
| 10 | 80.1 | 72.6 | 90.6 | 80.5 | 72.2 | 89.8 |
| 24 | 81.5 | 81.5 | 100.0 | 81.3 | 81.3 | 100.0 |
| 52 | 88.3 | 62.0 | 70.2 | 88.8 | 56.9 | 64.0 |

TABLE 2C
Glucose Isomerization at pH = 8

| | pH 8 | | |
|---|---|---|---|
| Time | Conv. | Yield | Sel. |
| (hr) | (%) | (%) | (%) |
| 2 | 25.9 | 29.3 | 113.0 |
| 4 | 49.8 | 52.3 | 105.2 |
| 6 | 67.1 | 62.5 | 93.0 |
| 8 | 73.4 | 73.3 | 99.9 |
| 10 | 79.9 | 74.3 | 93.0 |
| 24 | 82.0 | 82.0 | 100.0 |
| 52 | 88.3 | 63.4 | 71.8 |

In Tables 1, 2A, 2B, and 2C, the glucose to fructose percent conversion is evaluated by:

$$Percent Conversion of Glucose = \frac{Amount of glucose reacted}{Amount of initial glucose} \times 100 \%$$

Fructose yield is calculated by:

$$Fructose Yield = \frac{Amount of fructose produced}{Amount of initial glucose} \times 100 \%$$

The selectivity for fructose is calculated by:

$$Selectivity of Fructose = \frac{Amount of fructose produced}{Amount of glucose reacted} \times 100 \%$$

Experiment 2: Fructose Conversion to HMF

An experiment was conducted to evaluate the effect of reacting various MOFs with fructose to form HMF. Five trials were performed, and HMF selectivity and fructose conversion were measured and/or calculated for each trial. In each trial, different MOFs were used. The MOFs used for these trials included MIL101-Al—NH₂, uio-66-Zr—SO₃H, uio-66-Zr, MIL101-Cr—SO₃H, and MIL101-Cr. In each trial, 33 mg of fructose was reacted with 19 mg of the MOF catalyst in 0.67 g of [C₄mim]Cl solvent for 20 minutes at 100° C. The results are summarized in Table 3 below.

TABLE 3
Fructose Conversion using MOF

| | Fructose 100° C., 20 min | | | |
|---|---|---|---|---|
| | | HMF | | HMF |
| | Conversion | yield | HMF yield | selectivity |
| MOF | (wt. %) | (mol %) | (wt %) | (mol %) |
| MIL 101-Cr | 16.80 | 5.59 | 3.91 | 33.28 |
| MIL 101-Cr—SO₃H | 15.40 | 7.02 | 4.91 | 45.56 |
| uio-66-Zr | 32.20 | 16.11 | 11.28 | 50.03 |
| uio-66-Zr—SO₃H | 70.95 | 47.09 | 32.96 | 66.37 |
| MIL 101-Al—NH₂ | 13.14 | 9.91 | 6.94 | 75.44 |

Table 3 shows the percent conversion of fructose to HMF, HMF yield, and selectivity for HMF in each trial. The fructose to HMF percent conversion is evaluated by:

$$Percent Conversion of Fructose = \frac{Amount of fructose reacted}{Amount of initial fructose} \times 100 \%$$

HMF yield is calculated by:

$$HMF Yield = \frac{Amount of HMF produced}{Amount of initial fructose} \times 100 \%$$

The selectivity for HMF is calculated by:

$$Selectivity of HMF = \frac{Amount of HMF produced}{Amount of fructose reacted} \times 100 \%$$

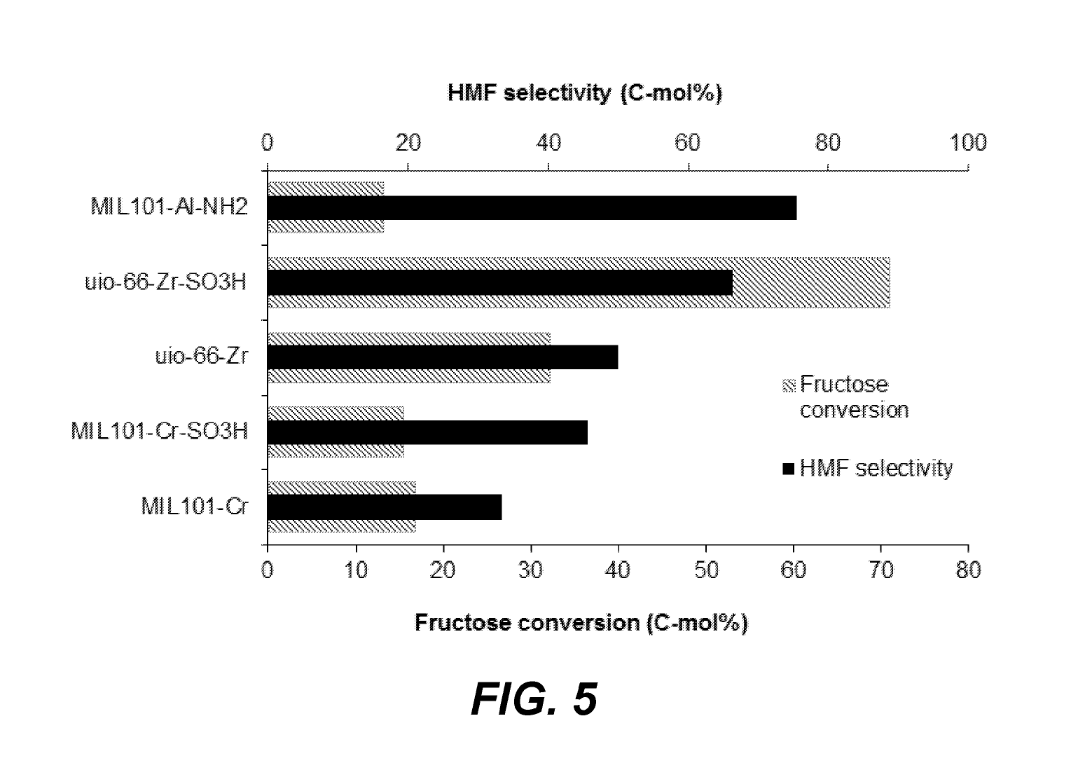

FIG. 5 shows a bar graph comparing HMF selectivity for each trial and fructose conversion for each trial. As indicated, the results show that MOFs effectively catalyzed fructose in [C₄mim]Cl with Al-MOF having the highest HMF selectivity. For Al-MOF in particular, HMF selectivity was high but fructose conversion was low. As a result, unreacted or unconverted fructose (about 80% of the initial fructose) may be recycled and reacted again with MOF after extracting out HMF to further drive formation of HMF.

FIG. 7 shows example structures of MOFs. In particular, the MIL-101 MOFs used in this experiment are depicted as the cage structures.

Experiment 3: Glucose Direct Conversion to HMF

An experiment was conducted to evaluate the effect of reacting various MOFs with glucose to form HMF. Five trials were performed, and HMF selectivity and fructose conversion were measured and/or calculated for each trial. In each trial, different MOFs were used. The MOFs used for these trials included MIL101-Al—NH₂, uio-66-Zr—SO₃H, uio-66-Zr, MIL101-Cr—SO₃H, and MIL101-Cr. In each trial, 33 mg of glucose was reacted with 19 mg of the MOF catalyst in 0.67 g of [C₄mim]Cl solvent for 120 minutes at 120° C. The results are summarized in Table 4 below.

TABLE 4
Glucose Conversion using MOF

| | Glucose 120° C., 120 min | | | |
|---|---|---|---|---|
| | | HMF | | HMF |
| | Conversion | yield | HMF yield | selectivity |
| MOF | (wt. %) | (mol %) | (wt. %) | (mol %) |
| MIL 101-Cr | 11.21 | 5.86 | 4.11 | 52.30 |
| MIL 101-Cr—SO₃H | 29.21 | 14.24 | 9.97 | 48.76 |
| uio-66-Zr | 49.17 | 9.63 | 6.74 | 19.59 |
| uio-66-Zr—SO₃H | 57.61 | 6.70 | 4.69 | 11.64 |
| MIL 101-Al—NH₂ | 5.31 | 4.18 | 2.92 | 78.63 |

Table 4 shows the percent conversion of glucose to HMF, HMF yield, and selectivity for HMF in each trial. The glucose to HMF percent conversion is evaluated by:

$$Percent Conversion of Glucose = \frac{Amount of glucose reacted}{Amount of initial glucose} \times 100 \%$$

HMF yield is calculated by:

$$HMF Yield = \frac{Amount of HMF produced}{Amount of initial glucose} \times 100 \%$$

The selectivity for HMF is calculated by:

$$Selectivity of HMF = \frac{Amount of HMF produced}{Amount of glucose reacted} \times 100 \%$$

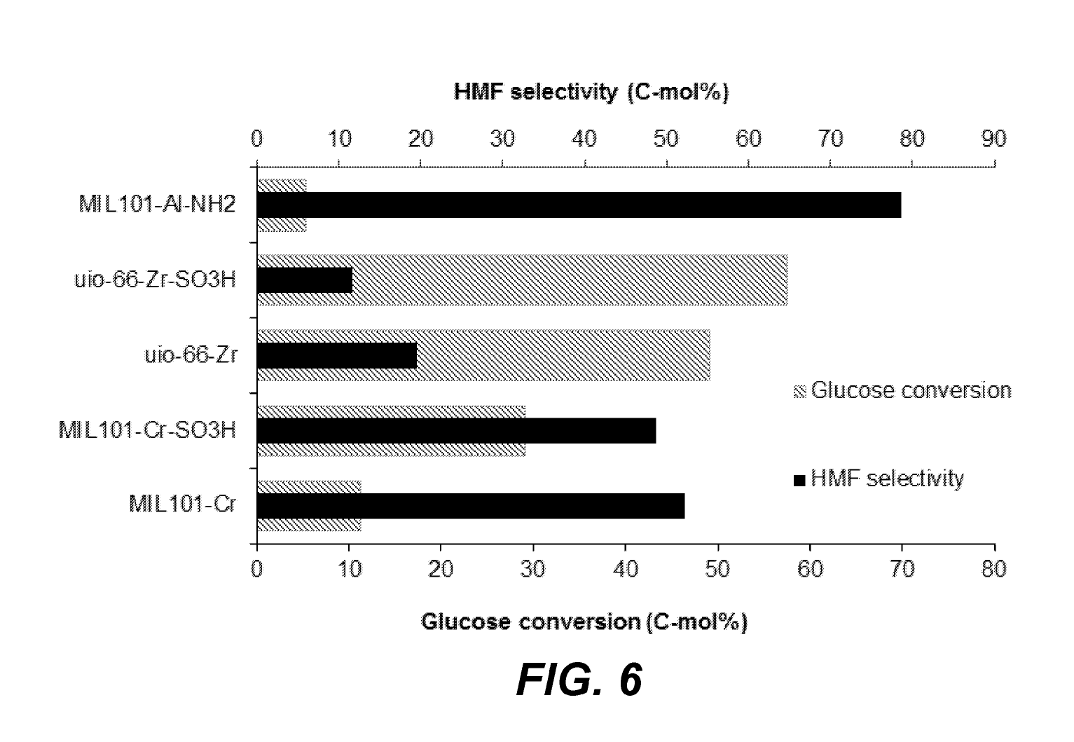

FIG. 6 shows a bar graph comparing HMF selectivity for each trial and glucose conversion for each trial. As indicated, the results show that MOFs effectively catalyzed glucose in [C₄mim]Cl with Al-MOF having the highest HMF selectivity. For Al-MOF in particular, HMF selectivity was high but glucose conversion was low. As a result, unreacted or unconverted glucose (about 95% of the initial glucose) may be recycled and reacted again with MOF after extracting out HMF to further drive formation of HMF.

FIG. 7 shows example structures of MOFs. In particular, the MIL-101 MOFs used in this experiment are depicted as the cage structures.

Experiment 4: Example Pathway From Cellulose to HMF

Experiments were conducted to measure HMF production using methods described in accordance with disclosed embodiments. 15 g of cellulose was pretreated with 85 g of [C₄mim]Cl at 140° C. for 1 hour with 15 wt % solid loading, followed by acid hydrolysis using HCl to convert cellulose to glucose. From 100 g of cellulose, 96.2 g of glucose and 10.2 g of HMF were formed.

In one trial, the glucose produced in the acidolysis reaction was then reacted with glucose isomerase and sodium borate with a mole ratio of glucose to borate of 1:0.5 at 70° C. for 10 hours. This isomerization yielded 76.6 g of fructose and 19.6 g of glucose. The fructose was then mixed with Al-MOF (MIL101-Al—NH₂) at 100° C. for 20 minutes to yield 8.0 g of HMF and 83.6 g of fructose. The fructose to HMF conversion reaction was performed without recycling any unreacted fructose. The reaction shows potential promise as the 83.6 g of fructose may be recycled to further react with Al-MOF and produce HMF.

In another trial, the glucose produced in the acidolysis reaction was directly reacted with Al-MOF (MIL101-Al—NH₂) at 120° C. for 2 hours to produce 91.1 g of glucose and 3.4 g of HMF. The reaction shows potential promise as the 91.1 g of glucose may be recycled to further react with Al-MOF and produce HMF.

Conclusion

Although the foregoing embodiments have been described in some detail for purposes of clarity of understanding, it will be apparent that certain changes and modifications may be practiced within the scope of the appended claims. It should be noted that there are many alternative ways of implementing the processes of the present embodiments. Accordingly, the present embodiments are to be considered as illustrative and not restrictive, and the embodiments are not to be limited to the details given herein.

1. A method of processing lignocellulose, the method comprising converting lignocellulosic derivatives to 5-hydroxymethyl furfural by reacting the lignocellulosic derivatives with a metal organic framework.

2. The method of claim 1, wherein the metal organic framework comprises aluminum.

3. The method of claim 1, wherein the metal organic framework comprises MIL-101.

4. The method of claim 1, wherein the lignocellulosic derivatives comprise glucose.

5. The method of claim 1, wherein the lignocellulosic derivatives comprise fructose.

6. The method of claim 1, further comprising prior to converting lignocellulosic derivatives to 5-hydroxymethyl furfural, forming the lignocellulosic derivatives from feedstock.

7. The method of claim 6, wherein forming lignocellulosic derivatives comprises acidolysis of cellulose to glucose.

8. The method of claim 6, wherein forming lignocellulosic derivatives comprises converting glucose to fructose using glucose isomerase and a borate salt.

9. The method of claim 6, wherein the method is performed in an acidic ionic liquid solvent.

10. The method of claim 9, wherein the acidic ionic liquid solvent is selected from the group consisting of [C₂mim]Cl, [C₃mim]Cl, and [C₄mim]Cl.

11. The method of claim 9, wherein forming the lignocellulosic derivatives and converting the lignocellulosic derivatives are performed in the same acidic ionic liquid solvent.

12. The method of claim 1, wherein the percent yield of 5-hydroxymethyl furfural is at least about 5% in weight.

13. The method of claim 1, wherein the selectivity for 5-hydroxymethylfurfural is at least about 70%.
